(19) United States
(12) Patent Application Publication
(10) Pub. No.: US 2007/0022498 A1
(43) Pub. Date: Jan. 25, 2007

(54) Corn plants having high oil and high protein content

(76) Inventors: Robert W. Pylman JR., Yamhill, OR (US); Robert H. Peterson, Lauderdale, MN (US); Charles M. Brown, Red Wing, MN (US)

Correspondence Address: JONDLE & ASSOCIATES P.C., 858 HAPPY CANYON ROAD SUITE 230, CASTLE ROCK, CO, 80108, US

(21) Appl. No.: 11/491,839

(22) Filed: Jul. 24, 2006

Related U.S. Application Data
(60) Provisional application No. 60/701,604, filed on Jul. 22, 2005.

(51) Int. Cl. A01H 1/00; A01H 5/00; C12N 15/82; C12N 5/04

(52) U.S. Cl. 800/281; 800/320.1; 435/412; 435/468; 800/284

(57) ABSTRACT

A corn seed having both high protein content and high oil content is provided. Methods for producing and using corn plants having both high protein content and high oil content are also provided. A corn seed having high protein content is provided.

This application claims the benefit of U.S. Provisional Application No. 60/701,604, filed Jul. 22, 2005 and which is hereby incorporated by reference.

### BACKGROUND

The present invention relates to corn plants having high oil and/or high protein content, to products obtained from these plants and to methods of using and producing these plants. All publications cited in this application are herein incorporated by reference.

Corn is an important crop used as a human food source, animal feed, and as a raw material in industry. The food uses of corn, in addition to the human consumption of corn kernels, include products of both the dry milling and wet milling industries. The principal products of dry milling include grits, meal and flour. The principal products of wet milling include starch, syrups, and dextrose. A by-product of both dry and wet milling is corn oil which is recovered from corn germ. As an animal feed, corn is used primarily as a feedstock for beef cattle, dairy cattle, swine, poultry, and fish.

Industrial uses of corn mainly consist of the use of corn starch produced by wet milling and corn flour produced by dry milling and whole kernel fermentation for production of food-grade and industrial use ethanol. The industrial applications of corn starch and flour are based on their functional properties, such as viscosity, film formation ability, adhesiveness, absorbent properties and ability to suspend particles. Corn starch and flour are used in the paper and textile industries and as components in adhesives, building materials, foundry binders, laundry starches, diapers, seed treatments, explosives, and oil-well muds. Plant parts other than the corn kernels are also used in industry. For example, stalks and husks can be made into paper and wallboard, and corn cobs can be used for fuel, to make charcoal and as a source of furfural.

Virtually all of the commercial corn produced in the United States is produced from hybrid seed. The production of hybrid seed first requires the development of elite corn inbred lines that possess good combining ability to produce agronomically superior hybrids. The majority of hybrid seed produced in the United States is of the single cross type, wherein two inbred lines are inter-mated, or crossed, to produce what is termed an F₁ single cross hybrid. The resulting kernels from this inter-mating are then sold as seed to commercial growers who plant the seed and harvest the second generation, or F₂ grain, for use on farm or for commercial sale.

There are numerous steps in the development of any novel, desirable plant germplasm. Plant breeding begins with the analysis and definition of problems and weaknesses of the current germplasm, the establishment of program goals, and the definition of specific breeding objectives. The next step is selection of germplasm that possess the traits to meet the program goals. The goal is to combine in a single variety or hybrid an improved combination of desirable traits from the parental germplasm. These important traits may include higher yield, resistance to diseases and insects, better stalks and roots, tolerance to drought and heat, reduction of grain moisture at harvest as well as better agronomic quality. With mechanical harvesting of many crops, uniformity of plant characteristics such as germination and stand establishment, growth rate, maturity and plant and ear height is important.

Choice of breeding or selection methods depends on the mode of plant reproduction, the heritability of the trait(s) being improved, and the type of cultivar used commercially (e.g., F₁ hybrid cultivar, pureline cultivar, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, recurrent selection, and backcross breeding.

The complexity of inheritance influences choice of breeding method. Backcross breeding is used to transfer one or a few favorable genes for a heritable trait into a desirable cultivar. This approach has been used extensively for breeding disease-resistant cultivars, nevertheless, it is also suitable for the adjustment and selection of morphological characters, color characteristics and simply inherited quantitative characters such as earliness, plant height or seed size and shape. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Each breeding program should include a periodic, objective evaluation of the efficiency of the breeding procedure. Evaluation criteria vary depending on the goal and objectives, but should include gain from selection per year based on comparisons to an appropriate standard, overall value of the advanced breeding lines, and number of successful cultivars produced per unit of input (e.g., per year, per dollar expended, etc.).

Promising advanced breeding lines are thoroughly tested per se and in hybrid combination and compared to appropriate standards in environments representative of the commercial target area(s) for three or more years. The best lines are candidates for use as parents in new commercial cultivars; those still deficient in a few traits may be used as parents to produce new populations for further selection.

These processes, which lead to the final step of marketing and distribution, usually take from eight to twelve years from the time the first cross is made. Therefore, development of new cultivars is a time-consuming process that requires precise forward planning, efficient use of resources, and a focus on clear objectives.

A most difficult task is the identification of individuals that are genetically superior, because for most traits the true genotypic value is masked by other confounding plant traits or environmental factors. One method of identifying a superior plant is to observe its performance relative to other experimental plants and to a widely grown standard cultivar. If a single observation is inconclusive, replicated observations provide a better estimate of its genetic worth.

The goal of corn breeding is to develop new, unique and superior corn inbred lines and hybrids. The breeder initially selects and crosses two or more parental lines, followed by repeated self pollination or selfing and selection, producing many new genetic combinations. The breeder can theoretically generate billions of different genetic combinations via crossing, selfing and mutations.

Each year, the plant breeder selects the germplasm to advance to the next generation. This germplasm is grown under unique and different geographical, climatic and soil conditions, and further selections are then made, during and at the end of the growing season. The inbred lines which are developed are unpredictable. This unpredictability is because the breeder's selection occurs in unique environments, with no control at the DNA level (using conventional breeding procedures), and with millions of different possible genetic combinations being generated. A breeder of ordinary skill in the art cannot predict the final resulting lines he develops, except possibly in a very gross and general fashion. This unpredictability results in the expenditure of large research funds to develop a superior new corn inbred line.

The development of commercial corn hybrids requires the development of homozygous inbred lines, the crossing of these lines, and the evaluation of the crosses. Pedigree breeding and recurrent selection breeding methods are used to develop inbred lines from breeding populations. Breeding programs combine desirable traits from two or more inbred lines or various broad-based sources into breeding pools from which inbred lines are developed by selfing and selection of desired phenotypes. The new inbreds are crossed with other inbred lines and the hybrids from these crosses are evaluated to determine which have commercial potential.

Pedigree breeding is used commonly for the improvement of self-pollinating crops or inbred lines of cross-pollinating crops. Two parents which possess favorable, complementary traits are crossed to produce an F₁. An F₂ population is produced by selfing one or several F₁s or by intercrossing two F₁s (sib mating). Selection of the best individuals is usually begun in the F₂ population; then, beginning in the F₃, the best individuals in the best families are selected. Replicated testing of families, or hybrid combinations involving individuals of these families, often follows in the F₄ generation to improve the effectiveness of selection for traits with low heritability. At an advanced stage of inbreeding (i.e., F₆ and F₇), the best lines or mixtures of phenotypically similar lines are tested for potential release as new cultivars.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding has been used to transfer genes for a simply inherited, highly heritable trait into a desirable cultivar or inbred line which is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

Descriptions of other breeding methods that are commonly used for different traits and crops can be found in one of several reference books (e.g., Principles of Plant Breeding, John Wiley and Son, pp. 115-161, 1960; Allard, 1960; Fehr, 1987).

Proper testing should detect any major faults and establish the level of superiority or improvement over current cultivars. In addition to showing superior performance, there must be a demand for a new cultivar that is compatible with industry standards or which creates a new market. The introduction of a new cultivar will incur additional costs to the seed producer, the grower, processor and consumer for special advertising and marketing, altered seed and commercial production practices, and new product utilization. The testing preceding release of a new cultivar should take into consideration research and development costs as well as technical superiority of the final cultivar. For seed-propagated cultivars, it must be feasible to produce seed easily and economically.

Once the inbreds that give the best hybrid performance have been identified, the hybrid seed can be reproduced indefinitely as long as the homogeneity of the inbred parent is maintained. A single-cross hybrid is produced when two inbred lines are crossed to produce the F₁ progeny. A double-cross hybrid is produced from four inbred lines crossed in pairs (A×B and C×D) and then the two F₁ hybrids are crossed again (A×B)×(C×D). Much of the hybrid vigor exhibited by F₁. hybrids is lost in the next generation (F₂). Consequently, seed from hybrid varieties is not used for planting stock.

Hybrid corn seed is typically produced by a male sterility system or by incorporating manual or mechanical detasseling. Alternate strips of two corn inbreds are planted in a field, and the pollen-bearing tassels are removed from one of the inbreds (female). Provided that there is sufficient isolation from sources of foreign corn pollen, the ears of the detasseled inbred will be fertilized only from the other inbred (male), and the resulting seed is therefore hybrid and will form hybrid plants.

The laborious, and occasionally unreliable, detasseling process can be avoided by using cytoplasmic male-sterile (CMS) inbreds. Plants of a CMS inbred are male sterile as a result of factors resulting from the cytoplasmic, as opposed to the nuclear, genome. Thus, this characteristic is inherited exclusively through the female parent in corn plants, since only the female provides cytoplasm to the fertilized seed. CMS plants are fertilized with pollen from another inbred that is not male-sterile. Pollen from the second inbred may or may not contribute genes that make the hybrid plants male-fertile. Seed from detasseled fertile corn and CMS produced seed of the same hybrid can be blended to insure that adequate pollen loads are available for fertilization when the hybrid plants are grown.

There are several methods of conferring genetic male sterility available, such as multiple mutant genes at separate locations within the genome that confer male sterility, as disclosed in U.S. Pat. Nos. 4,654,465 and 4,727,219 to Brar et al. These and all patents referred to are incorporated by reference. In addition to these methods, Albertsen et al., U.S. Pat. No. 5,432,068 have developed a system of nuclear male sterility which includes: identifying a gene which is critical to male fertility, silencing this native gene which is critical to male fertility; removing the native promoter from the essential male fertility gene and replacing it with an inducible promoter; inserting this genetically engineered gene back into the plant; and thus creating a plant that is male sterile because the inducible promotyer is not “on” resulting in the male fertility gene not being transcribed. Fertility is restored by inducing, or turning “on”, the promoter, which in turn allows the gene that confers male fertility to be transcribed.

There are many other methods of conferring genetic male sterility in the art, each with its own benefits and drawbacks. These methods use a variety of approaches such as delivering into the plant a gene encoding a cytotoxic substance associated with a male tissue specific promoter or an anti-sense system in which a gene critical to fertility is identified and an antisense to that gene is inserted in the plant (see, Fabinjanski, et al. EPO 89/0301053.8 publication no. 329,308 and PCT application PCT/CA90/00037 published as WO 90/08828).

Another version useful in controlling male sterility makes use of gametocides. Gametocides are not a genetic system, but rather a topical application of chemicals. These chemicals affect cells that are critical to male fertility. The application of these chemicals affects fertility in the plants only for the growing season in which the gametocide is applied (see Carlson, G. R., U.S. Pat. No. 4,936,904). Application of the gametocide, timing of the application, and genotype often limit the usefulness of the approach.

The objective of typical plant breeding is to combine in a single variety or hybrid the desirable traits of the parental lines. For field crops such as corn, these desirable traits may include resistance to diseases, insects, herbicide tolerance, and tolerance to heat and drought, reduced time to crop maturity, and improved agronomic quality, such as high oil content and high protein content. With mechanical harvesting of many crops, uniformity of plant characteristics such as germination time and stand establishment, growth rate, and fruit/seed size are also desirable.

There exists a need for corn having high oil, increased protein and other special end-use properties which is not met by current corn inbreds or hybrids. The foregoing examples of the related art and limitations related therewith are intended to be illustrative and not exclusive. Other limitations of the related art will become apparent to those of skill in the art upon a reading of the specification and a study of the drawings.

### SUMMARY OF THE INVENTION

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

It is an aspect of the present invention to provide corn plants which produce grain having both high oil and high protein content.

It is another aspect of the present invention to provide corn plants which produce grain having high protein content.

It is another aspect of the present invention to provide corn plants which produce grain having both high oil and high protein content, wherein said grain is used for animal feed.

It is another aspect of the present invention to provide corn plants which produce grain having both high oil and high protein content, wherein said grain is used for human consumption.

It is another aspect of the present invention to provide corn plants which produce grain having both high oil and high protein content, wherein said grain is used to provide biofuel.

It is another aspect of the present invention to provide corn plants which produce grain having both high oil and high protein content, wherein said grain is used for the production of ethanol.

It is another aspect of the present invention to provide a corn plant produced by the hybrid method wherein at least one parent is a corn plant that produces grain having both high oil and high protein content.

In addition to the exemplary aspects and embodiments described above, further aspects and embodiments will become apparent by study of the following descriptions.

### Definitions

In the description and tables which follow, a number of terms are used. In order to provide a clear and consistent understanding of the specification and claims, including the scope to be given such terms, the following definitions are provided:

Allele. The “allele” is any of one or more alternative form of a gene, all of which alleles relates to one trait or characteristic. In a diploid cell or organism, the two alleles of a given gene occupy corresponding loci on a pair of homologous chromosomes.

Anthesis. “Anthesis” is the period or act of flowering.

Backcross. “Backcross” is the cross of a hybrid to either one of its parents. The offspring of such a cross is referred to as the backcross generation.

Btu/Lb. “Btu” is short for British thermal unit, a standard unit of energy. One Btu is equal to the amount of heat required to raise the temperature of one pound of liquid water by 1 degree Fahrenheit at its maximum density, which occurs at a temperature of 39.1 degrees Fahrenheit. One Btu is equal to approximately 251.9 calories or 1055 joules. “Btu/Lb” is the amount of energy generated by a particular quantity of matter.

Commercially acceptable. “Commercially acceptable” is any corn hybrid that is sold commercially.

Daily heat unit value. The “daily heat unit value” is calculated as follows: (the maximum daily temperature+the minimum daily temperature)/2 minus 50. All temperatures are in degrees Fahrenheit. The maximum temperature threshold is 86 degrees, if temperatures exceed this, 86 is used. The minimum temperature threshold is 50 degrees, if temperatures go below this, 50 is used.

Donor parent. A “donor parent” is the parent from which one or a few genes are transferred to the recurrent parent in backcross breeding.

Dropped Ears. “Dropped ears” is a measure of the number of dropped ears per plot, and represents the percentage of plants that dropped an ear prior to harvest.

Dry down. “Dry down” is the rate at which a hybrid will reach acceptable harvest moisture.

Ear Height. The “ear height” is a measure from the ground to the ear node attachment, and is measured in centimeters.

Embryo. The “embryo” is the rudimentary plant in a seed. The embryo arises from the zygote.

Endosperm. The “endosperm” is the nutritive tissue formed within the embryo sac in seed plants. It commonly arises following the fertilization of the two primary endosperm nuclei of the embryo sac by the two male sperm. In a diploid organism the endosperm is triploid.

Essentially all the physiological and morphological characteristics. A plant having “essentially all the physiological and morphological characteristics” means a plant having the physiological and morphological characteristics of the recurrent parent, except for the characteristics derived from the converted gene.

GDU Pollen. “GDU pollen” is the number of heat units from planting until 50% of the plants in the hybrid are shedding pollen.

GDU Silk. The “GDU silk” (=heat unit silk) is the number of growing degree units (GDU) or heat units required for an inbred line or hybrid to reach silk emergence from the time of planting. Growing degree units are calculated by the Barger Method, where the heat units for a 24-hour period are: GDU=((Max Temp+Min Temp)/2)-50 The highest maximum used is 86° F. and the lowest minimum used is 50° F. For each hybrid, it takes a certain number of GDUs to reach various stages of plant development. GDUs are a way of measuring plant maturity.

Harvest Aspect. “Harvest aspect” is a visual rating given the day of harvest or the previous day. Hybrids are rated 1 (poorest) to 9 (best) with poorer scores given for poor plant health, visible signs of fungal infection, poor plant intactness characterized by missing leaves, tassels, or other vegetative parts, or a combination of these traits.

High oil. “High oil” refers to corn having an oil content greater than 6.5%.

High protein. “High protein” refers to corn having a protein content greater than 7.5%.

HTU. “HTU” is the summation of the daily heat unit value calculated from planting to harvest.

Kernel. The “kernel” is the corn caryopsis comprising a mature embryo and endosperm which are products of double fertilization.

Late plant greenness. “Late plant greenness” is a visual assessment given at around the dent stage but typically a few weeks before harvest to characterize the degree of greenness left in the leaves. Plants are rated from 1 (poorest) to 9 (best) with poorer scores given for plants that have more non-green leaf tissue typically due to early senescence or from disease.

Male sterility. “Male sterility” is a condition in which pollen is absent or non-functional in flowering plants.

Moisture. The “moisture” is the actual percentage moisture of the grain at harvest.

Oil content. This is the oil concentration of a corn kernel expressed on a dry weight basis.

Percent protein. This is the total protein concentration of a corn kernel expressed on a dry weight basis.

Percent oil. This is the oil concentration of a corn kernel expressed on a dry weight basis.

Plant Cell. “Plant cell”, as used herein, includes plant cells whether isolated, in tissue culture or incorporated in a plant or plant part.

Plant habit. “Plant habit” is a visual assessment assigned during the late vegetative to early reproductive stages to characterize the plant's leaf habit. It ranges from decumbent with leaves growing horizontally from the stalk to a very upright leaf habit, with leaves growing near vertically from the stalk.

Plant Height. “Plant height” is a measure of the height of the plant from the ground to the tip of the tassel, and is measured in centimeters.

Plant intactness. “Plant intactness” is a visual assessment assigned to a hybrid or inbred at or close to harvest to indicate the degree that the plant has suffered disintegration through the growing season. Plants are rated from 1 (poorest) to 9 (best) with poorer scores given for plants that have more of their leaf blades missing.

Plant Part. As used herein, the term “plant part” includes leaves, stems, roots, seed, grain, embryo, pollen, ovules, flowers, ears, cobs, husks, stalks, root tips, anthers, silk, tissue, cells and the like.

Pollen shed. “Pollen shed” is a visual rating assigned at flowering to describe the abundance of pollen produced by the anthers. Plants are rated 1 (poorest) to 9 (best) with the best scores for plants with tassels that shed more pollen during anthesis.

Pollinators. “Pollinators” are male fertile corn plants used to pollinate male sterile corn plants.

Post-anthesis Root Lodging. “Post-anthesis root lodging” is a percentage of plants that root lodge after anthesis, that is, plants that lean from the vertical axis at an approximately 30° angle or greater.

Pre-anthesis Brittle Snapping. “Pre-anthesis brittle snapping” is a percentage of “snapped” plants following severe winds prior to anthesis.

Pre-anthesis Root Lodging. “Pre-anthesis root lodging” is a percentage of plants that root lodge prior to anthesis, that is, plants that lean from the vertical axis at an approximately 30° angle or greater.

Protein content. “Protein content” is the total protein concentration of a corn kernel expressed on a dry weight basis.

Quantitative Trait Loci (QTL). “Quantitative trait loci” refers to genetic loci that control to some degree numerically representable traits that are usually continuously distributed.

Recurrent parent. “Recurrent parent” refers to the parent to which the first cross and successive backcrossed plants are crossed.

Regeneration. “Regeneration” refers to the development of a plant from tissue culture.

Root Lodging. “Root lodging” is the percentage of plants that root lodge, that is, those that lean from the vertical axis at an approximately 30° angle or greater would be counted as root lodged.

Seed quality. “Seed quality” is a visual rating assigned to the kernels of the plant. Kernels are rated 1 (poorest) to 9 (best) with poorer scores given for kernels that are very soft and shriveled with splitting of the pericarp visible and better scores for fully formed kernels.

Seedling Vigor. “Seedling vigor” is the vegetative growth after emergence at the seedling stage at approximately five leaves.

Single cross. A “single cross” is a cross between two genotypes, usually two genetically different inbred lines or synthetic lines.

Single gene converted. “Single gene converted” or “conversion” plant refers to plants which are developed by a plant breeding technique called backcrossing wherein essentially all the morphological and physiological characteristics of an inbred are recovered in addition to the single gene transferred into the inbred via the backcrossing technique or via genetic engineering.

Stalk Lodging. “Stalk lodging” is the percentage of plants that stalk lodge, i.e., stalk breakage, as measured by either natural lodging or by pushing the stalks to determine the percentage of plants that break off below the ear. This is a relative rating of a plant to other plants for standability.

Standability. “Standability” refers to how well a plant remains upright towards the end of the growing season. Plants with excessive stalk breakage and/or root lodging would be considered to have poor standability.

Stay Green. “Stay green” is the measure of plant health near the time of black layer formation (physiological maturity). A high score indicates better late-season plant health.

TOPCROSS. “TOPCROSS” is a method (U.S. Pat. No. 5,706,603) for breeding corn in which approximately 90-95% of the corn plants in the planted acreage are male sterile grain parents and approximately 5-10% of the corn plants in the planted acreage are pollinator plants.

Variety. A plant “variety” as used by one skilled in the art of plant breeding means a plant grouping within a single botanical taxon of the lowest known rank which can be defined by the expression of the characteristics resulting from a given genotype or combination of phenotypes, distinguished from any other plant grouping by the expression of at least one of the said characteristics and considered as a unit with regard to its suitability for being propagated unchanged. (International Convention for the Protection of New Varieties of Plants)

Yield (Bushels/Acre). The “yield” is the actual yield of the grain at harvest adjusted to 15.5% moisture.

### DETAILED DESCRIPTION OF THE INVENTION

Prior to the instant invention, hybrid corn plants that are commercially acceptable have never been developed having both high oil content and high protein content combined. These traits have not previously been combined in any commercial or wild type corn plants. In addition, it was quite unexpectedly found that the high oil and high protein traits appear to have a strong dominant effect in crossing and segregation.

An important aspect of the present invention is that it can be used for animal feed because it provides corn plants which produce grain having both high oil and high protein content. Corn silage is recognized as an excellent forage for cattle because it is high yielding, palatable, and a good source of feed energy. Its energy content makes it particularly desirable as the major feed-stuff in rations for growing calves and as the roughage for finishing cattle. A disadvantage of conventional corn silage is that it is relatively low in protein. Thus, supplemental protein is needed in most corn-based backgrounding and finishing diets, because of conventional corn's low crude protein content. The grain produced by the present invention, having higher protein content than conventional corn grain, obviates the need for protein supplementation when used as animal feed. Because fewer supplements would be required in rations to meet the nutritional requirements of the animal, the use of the present invention would also be more economical for the livestock producer than the use of conventional corn.

Another advantage the present invention provides to the livestock producer is the higher oil content of the corn grain. Corn oil has twice as much energy as starch, on a per unit weight basis, so high oil corn has more nutrient or caloric density than regular corn. For the livestock producer, the higher caloric density provides benefits in two ways: 1) It can substitute for fat already being added to optimized rations; and 2) it can add energy to rations already being optimized. The higher caloric density means animals will get more growth or production energy for any given volume of feed consumed. High oil corn also offers a better amino acid balance than normal corn, with somewhat higher levels of lysine and methionine. In operations where dust levels are a problem (feed mills, confinement facilities, etc.) high oil corn offers the additional advantage of low dust, eliminating the need for spraying with water or vegetable oil to reduce dust. (U.S. Pat. No. 5,750,851). The corn grain of the present invention, having higher oil content than conventional corn grain and having higher protein content than conventional corn grain, provides both higher caloric density and lower dust than conventional corn grain.

The present invention encompasses corn seed having oil content of 6.5%, 6.6%, 6.7%, 6.8%, 6.9%, 7.0%, 7.1%, 7.2%, 7.3%, 7.4%, 7.5%, 7.6%, 7.7%, 7.8%, 7.9% 8.0%, 8.1%, 8.2%, 8.3%, 8.4%, 8.5%, 8.6%, 8.7%, 8.8%, 8.9%, 9.0%, 9.1%, 9.2% 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, 13.0%, 13.1%, 13.2%, 13.3%, 13.4%, 13.5%, 13.6%, 13.7%, 13.8%, 13.9%, 14.%, 14.1%, 14.5%, 14.6%, 14.7%, 14.8%, 14.9%, 15.0%, 15.1%, 15.2%, 15.3%, 15.4%, 15.5%, 15.6%, 15.7%, 15.8%, 15.9%, 16.0%, 16.1%, 16.2%, 16.3%, 16.4%, 16.5%, 16.6%, 16.7%, 16.8%, 16.9%, 17.0%, 17.1%, 17.2%, 17.3%, 17.4%, 17.5%, 17.6%, 17.7%, 17.8%, 17.9%, 18.0%, 18.1%, 18.2%, 18.3%, 18.4%, 18.5%, 18.6%, 18.7%, 18.8%, 18.9%, 19.0%, 19.1%, 19.2%, 19.3%, 19.4%, 19.5%, 19.6%, 19.7%, 19.8%, 19.9%, 20.0%, 20.1%, 20.2%, 20.3%, 20.4%, 20.5%, 20.6%, 20.7%, 20.8%, 20.9%, 21.0%, 21.1%, 21.2%, 21.3%, 21.4%, 21.5%, 21.6%, 21.7%, 21.8%, 21.9%, 22.0%, 22.1%, 22.2%, 22.3%, 22.4%, 22.5%, 22.6%, 22.7%, 22.8%, 22.9%, 23.0%, 23.1%, 23.2%, 23.3%, 23.4%, 23.5%, 23.6%, 23.7%, 23.8%, 23.9%, 24.0% and all fractions thereof.

The present invention encompasses corn seed having protein content of 7.5%, 7.6%, 7.7%, 7.8%, 7.9%, 8.0%, 8.1%, 8.2%, 8.3%, 8.4%, 8.5%, 8.6%, 8.7%, 8.9%, 9.0%, 9.1%, 9.2%, 9.3%, 9.4%, 9.5%, 9.6%, 9.7%, 9.8%, 9.9%, 10.0% 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3%, 14.4%, 14.5%, 14.6%, 14.7%, 14.8%, 14.9%, 15.0%, 15.1%, 15.2%, 15.3%, 15.4%, 15.5%, 15.6%, 15.7%, 15.8%, 15.9%, 16.0%, 16.1%, 16.2%, 16.3%, 16.4%, 16.5%, 16.6%, 16.7%, 16.8%, 16.9%, 17.0%, 17.1%, 17.2%, 17.3%, 17.4%, 17.5%, 17.6%, 17.7%, 17.8%, 17.9%, 18.0% and all fractions thereof.

The present invention encompasses corn seed, when used for fuel in combustion, producing Btu/lb in the range of 8450, 8475, 8500, 8525, 8550, 8575, 8600, 8625, 8650, 8675, 8700, 8725, 8750, 8775, 8800, 8825, 8850, 8875, 8900, 8925, 8950, 8975, 9000, 9025, 9050, 9075, 9100, 9125, 9150, 9175, 9200 and all fractions thereof.

Another important aspect of the present invention is that corn grain having both high oil and high protein content provides a better biofuel than conventional corn. For the biomass fuel user, the higher caloric density provides benefits in two ways: 1) more heat may be extracted for any given volume of high oil corn than conventional corn and 2) in the home, where dust levels are a concern, high oil corn offers the additional advantage of low dust. The corn grain of the present invention, having higher oil content than conventional corn grain and having higher protein content than conventional corn grain, provides both higher caloric density and lower dust than conventional corn grain.

Another important aspect of the present invention is that the high oil, high protein grain could be used for the production of ethanol. Currently corn is used for the production of ethanol, an important fuel and industrial chemical. However, the present invention yields a greater quantity of ethanol than conventional corn.

An important aspect of the present invention is that the high oil, high protein grain produced by the present invention could be used for human consumption. For the food ingredient manufacturer high oil corn allows more oil to be obtained from less grain than conventional corn. High oil corn also allows more than one type of corn meal to be prepared from a single type of corn (U.S. Patent Application 20040058052). In addition, corn with higher protein content provides a more nutritious food product. The corn plants of the present invention, producing grain having higher oil content than conventional corn plants and producing grain having higher protein content than conventional corn plants, provide both higher caloric density and greater nutrition than conventional corn plants for human consumption.

### EXAMPLES

### Example 1

### Inbred Corn Line 3B703

Inbred corn line 3B703 is a yellow field corn which provides an excellent parental line in crosses for producing high oil, high protein first generation (F₁) hybrid corn.

Some of the criteria used to select ears in various generations include: yield, percentage moisture, percentage oil, percentage protein, stalk quality, root quality, disease tolerance, late plant greenness, late season plant intactness, tip fill, barrenness, ear shape and size, seed quality, ear height, pollen shedding ability, and corn borer tolerance. During the development of the line, crosses were made to inbred testers for the purpose of estimating the line's general and specific combining ability. The inbred was then evaluated further as a line and in numerous crosses. Unexpectedly, it was found that the high oil and high protein traits appear to have a strong dominant effect in crossing and segregation. Inbred corn line 3B703 has the following morphologic and other characteristics.

TABLE 1
VARIETY DESCRIPTION INFORMATION

| | |
|---|---|
| Plant: | a. Average number of tillers: 0 |
| | b. Average number of ears per stalk: 1.3 |
| | c. Anthocyanin of brace roots: Faint |
| Maturity: | d. Days to 50% Flower: 94 |
| | e. GDU to 50% Flower: 1291 |
| | f. Days to 50% Silk: 95 |
| | g. GDU to 50% Silk: 1315 |
| Tassel: | h. Pollen shed: 9 (rated on a scale of 0 = male sterile |
| | to 9 = heavy shed) |
| Kernel (dried): | i. Length: 8.3 mm |
| | j. Kernel width: 6.8 mm |
| | k. Kernel thickness: 5.3 mm |
| | l. Weight per 100 Kernels: 13.85 g |
| Agronomic traits: | m. Stay green (at 65 days after anthesis: 8 (rated on |
| | a scale of 1 = worst to 9 = excellent) |
| | n. Pre-anthesis root lodging: 0% |
| | o. Post-anthesis root lodging: 2% |
| Disease and Pest | p. Resistance to Fusarium: 7 (rated on a scale of |
| Resistance: | 1 = susceptible to 9 = resistant) |
| | q. Resistance to Eyespot: 5 (rated on a scale of |
| | 1 = susceptible to 9 = resistant) |

In Table 2 below, the grain percent protein (dry basis) and percent oil (dry basis) from three corn hybrids produced by using corn inbred 3B703 of the present invention as a hybrid parent are presented. These three corn hybrids, LH176RR×LH177RR×3B703, LH227RR×3B703, and LH245RR×3B703, were produced using standard hybrid breeding methods (NOT by using the TOPCROSS method). Column 1 shows the location where the hybrids were grown, where WI is Wisconsin, MI is Michigan, and MN is Minnesota. Column 2 shows the pedigree and columns 3 through 5 show the percent moisture, percent protein (dry basis) and percent oil (dry basis) respectively. [(LH74×LH146)×LH163]-P41 is a TOPCROSS blend for comparison, where male sterile hybrid [(LH74×LH146)×LH163] is grown in a field with pollinator line P41.

As shown in table 2 below, the level of protein and the level of oil in combination were unexpectedly high in the hybrids of the present invention. Increased oil content in corn often results in decreased protein content. Likewise, increased protein content often results in decreased oil content. However, the present invention results in corn plants that maintain high levels of both protein and oil.

TABLE 2

| Loc | Pedigree | % Moisture | % Protein (DB) | % Oil (DB) |
|---|---|---|---|---|
| WI | LH176RR × LH177RR)(3B703 | 23.41 | 10.44 | 10.36 |
| WI | LH176RR × LH177RR)(3B703 | 6.31 | 13.01 | 9.16 |
| MI | LH176RR × LH177RR)(3B703 | 29.1 | 11.5 | 7.1 |
| MI | LH176RR × LH177RR)(3B703 | 26.9 | 11.0 | 10.1 |
| WI | LH227RR × 3B703 | 27.89 | 11.36 | 9.27 |
| WI | LH227RR × 3B703 | 5.42 | 14.18 | 10.78 |
| MI | LH227RR × 3B703 | 24.7 | 13.3 | 10.3 |
| MI | LH227RR × 3B703 | 28.8 | 11.0 | 10.9 |
| MI | LH227RR × 3B703 | 25.9 | 9.0 | 9.8 |
| WI | LH245RR × 3B703 | 24.42 | 9.33 | 10.62 |
| WI | LH245RR × 3B703 | 6.09 | 13.29 | 10.35 |
| MI | LH245RR × 3B703 | 23.6 | 11.3 | 8.6 |
| MI | LH245RR × 3B703 | 27.3 | 9.8 | 9.1 |
| MI | LH245RR × 3B703 | 31.5 | 11.3 | 9.5 |
| MI | LH245RR × 3B703 | 29.1 | 9.6 | 8.6 |
| Ml | HC53 × HE323 | 23.7 | 7.9 | 4.7 |
| MI | HC53 × HE323 | 26.8 | 7.2 | 5.7 |
| MI | HC53 × HE323 | 26.9 | 7.1 | 5.8 |
| MI | HC53 × LH290 | 29.0 | 9.8 | 4.6 |
| MI | HC53 × LH290 | 22.2 | 9.2 | 5.4 |
| MI | HC53 × LH290 | 21.3 | 9.2 | 5.1 |
| WI | [(LH74 × LH146) × LH163] − P41 | 29.92 | 8.42 | 3.83 |
| WI | [(LH74 × LH146) × LH163] − P41 | 29.42 | 8.61 | 4.23 |
| MN | [(LH74 × LH146) × LH163] − P41 | 12.37 | 9.94 | 7.61 |
| MN | [(LH74 × LH146) × LH163] − P41 | 13.72 | 10.57 | 7.22 |
| MN | [(LH74 × LH146) × LH163] − P41 | 16.04 | 10.02 | 6.91 |
| MN | [(LH74 × LH146) × LH163] − P41 | 10.89 | 11.09 | 8.34 |

In table 3 below, the energy generated (Btu/Lb) by hybrids produced by using inbred corn line 3B703 as a hybrid parent is compared with those generated by two conventional hybrid corn plants. As shown in table 3, the energy generated by the hybrids produced by using inbred corn line 3B703 as a hybrid parent is greater than that generated by the two conventional hybrid corn plants. The seeds were used to produce the energy. The increase in energy generated ranged from 277 Btus per pound to 443 Btus per pound over that generated by the conventional hybrid corn plants. This was a greater increase in energy output over the conventional hybrid corn plants than had been expected.

TABLE 3

| Corn Plant | BTU/LB |
|---|---|
| LH176RR × LH177RR)(3B703 | 8535 |
| LH227RR × 3B703 | 8497 |
| LH245RR × 3B703 | 8622 |
| Christian Hybrid-Blooming Prairie (Conventional) | 8220 |
| Bird Island Seed Hybrid (Conventional) | 8179 |

### Example 2

### Inbred Corn Line 3B603

Inbred corn line 3B603 is a yellow field corn which provides an excellent parental line in crosses for producing high oil, high protein first generation (F₁) hybrid corn.

Some of the criteria used to select ears in various generations include: yield, percentage moisture, percentage oil, percentage protein, stalk quality, root quality, disease tolerance, late plant greenness, late season plant intactness, tip fill, barrenness, ear shape and size, seed quality, ear height, pollen shedding ability, and corn borer tolerance. During the development of the line, crosses were made to inbred testers for the purpose of estimating the line's general and specific combining ability. The inbred was then evaluated further as a line and in numerous crosses. Unexpectedly, it was found that the high oil and high protein traits appear to have a strong dominant effect in crossing and segregation. Inbred corn line 3B603 has the following morphologic and other characteristics.

TABLE 4
VARIETY DESCRIPTION INFORMATION

| | |
|---|---|
| Plant: | a. Average number of tillers: 0 |
| | b. Average number of ears per stalk: 1.3 |
| | c. Anthocyanin of brace roots: moderate |
| Maturity: | a. Days to 50% Flower: 91 |
| | b. GDU to 50% Flower: 1229 |
| | c. Days to 50% Silk: 93 |
| | d. GDU to 50% Silk: 1278 |
| Tassel: | a. Number of lateral branches: 14 |
| | b. Pollen shed: 9 (Rated on a scale of 0 = male sterile |
| | to 9 = heavy shed) |
| | c. Glume color: Light green |
| | d. Bar glumes: None |
| Kernel (dried): | a. Kernel length: 7.6 mm |
| | b. Kernel width: 6.5 mm |
| | c. Kernel thickness: 4.4 mm |
| | d. Weight per 100 kernels: 11.29 g |
| Agronomic traits: | a. Stay green (at 65 days after anthesis): 5 (rated on a |
| | scale of 1 = worst to 9 = excellent) |
| | b. Dropped ears (at 65 days after anthesis): 0% |
| | c. Pre-anthesis root lodging: 2% |
| | d. Post-anthesis root lodging: 4% |
| Disease and pest | a. Resistance to Fusarium: 7 (rated on a scale of |
| resistance: | 1 = susceptible to 9 = resistance) |
| | b. Resistance to Eyespot: 3 (rated on a scale of |
| | 1 = susceptible to 9 = resistant) |

In table 5 below, the grain percent protein (dry basis) and percent oil (dry basis) from two corn hybrids produced by using corn inbred 3B603 of the present invention as a hybrid parent are presented. These hybrids, LH227RR×3B603 and 3B50HO×3B603, were produced using standard hybrid breeding methods (NOT by using the TOPCROSS method). Column 1 shows the pedigree and columns 2 through 4 show the percent moisture, percent protein (dry basis) and percent oil (dry basis) respectively. HC53×LH290 and HC53×LHE323 are conventional hybrids for comparison. All the hybrids were grown in Michigan.

As shown in table 5 below, the level of protein and the level of oil in combination were unexpectedly high in the hybrids of the present invention. Normally, increased oil content in corn results in decreased protein content while increased protein content results in decreased oil content. However, the present invention has resulted in corn plants that maintain high levels of both protein and oil. Additionally, the results indicate the strong dominate effect of the high oil, high protein traits in the hybrids.

TABLE 5

| Pedigree | % Moisture | % Protein (DB) | % Oil (DB) |
|---|---|---|---|
| LH227RR × 3B603 | 26.6 | 9.9 | 8.8 |
| LH227RR × 3B603 | 28.0 | 9.7 | 8.8 |
| LH227RR × 3B603 | 28.7 | 10.9 | 9.7 |
| LH227RR × 3B603 | 31.0 | 10.1 | 9.6 |
| 3B50HO × 3B603 | 27.3 | 9.2 | 8.5 |
| 3B50HO × 3B603 | 28.9 | 9.4 | 9.0 |
| 3B50HO × 3B603 | 26.7 | 9.8 | 10.2 |
| HC53 × LHE323 | 23.7 | 7.9 | 4.7 |
| HC53 × LHE323 | 26.8 | 7.2 | 5.7 |
| HC53 × LHE323 | 26.9 | 7.1 | 5.8 |
| HC53 × LH290 | 29.0 | 9.8 | 4.6 |
| HC53 × LH290 | 21.3 | 9.2 | 5.1 |
| HC53 × LH290 | 22.2 | 9.2 | 5.4 |

In table 6 below, the energy generated (Btu/Lb) by hybrids produced by using inbred corn line 3B603 as a hybrid parent is compared with those generated by two conventional hybrid corn plants. As shown in table 6, the energy generated by the hybrids produced by using inbred corn line 3B603 as a hybrid parent is greater than that generated by the two conventional hybrid corn plants. The seeds were used to produce the energy. The increase in energy generated ranged from 284 Btus per pound to 843 Btus per pound over that generated by the conventional hybrid corn plants. This was a greater increase in energy output over the conventional hybrid corn plants than had been expected.

TABLE 6

| Corn Plant | BTU/LB |
|---|---|
| 3B75 × 3B603 | 8504 |
| 3B60 × 3B603 | 9022 |
| Christian Hybrid-Blooming Prairie (Conventional) | 8220 |
| Bird Island Seed Hybrid (Conventional) | 8179 |

### Example 3

### Inbred Corn Line 3B50HO

Inbred corn line 3B50HO is a yellow field corn which provides an excellent parental line in crosses for producing high oil, high protein first generation (F₁) hybrid corn.

Some of the criteria used to select ears in various generations include: yield, percentage moisture, percentage oil, percentage protein, stalk quality, root quality, disease tolerance, late plant greenness, late season plant intactness, tip fill, barrenness, ear shape and size, seed quality, ear height, pollen shedding ability, and corn borer tolerance. During the development of the line, crosses were made to inbred testers for the purpose of estimating the line's general and specific combining ability. The inbred was then evaluated further as a line and in numerous crosses. Unexpectedly, it was found that the high oil and high protein traits appear to have a strong dominant effect in crossing and segregation. Inbred corn line 3B50HO has the following morphologic and other characteristics.

TABLE 7
VARIETY DESCRIPTION INFORMATION

| | |
|---|---|
| Plant: | a. Average number of tillers: 0 |
| | b. Average number of ears per stalk: 1.2 |
| | c. Anthocyanin of brace roots: Moderate |
| Leaf: | a. Width of ear node leaf: 10.1 cm |
| | b. Length of ear node leaf: 77.4 cm |
| | c. Number of leaves above top ear: 5.0 |
| | d. Leaf color: Light green |
| | e. Leaf sheath pubescence: 1.2 (Rated on a scale of |
| | 1 = none to 9 = like peach fuzz) |
| | f. Marginal Waves: 2.4 (Rated on a scale of |
| | 1 = none to 9 = many) |
| | g. Longitudinal creases: 1.3 (Rated on a scale of |
| | 1 = none to 9 = many) |
| Maturity: | a. Days to 50% Flower: 80 |
| | b. GDU to 50% Flower: 1061 |
| | c. Days to 50% Silk: 81 |
| | d. GDU to 50% Silk: 1086 |
| Tassel: | a. Number of lateral branches: 18 |
| | b. Tassel length (from top leaf collar to tassel top): |
| | 36 cm |
| | c. Pollen shed: 8 (Rated on a scale of 0 = male sterile |
| | to 9 = heavy shed) |
| | d. Anther color: Purple |
| | e. Glume color: Green |
| | f. Bar glumes: Present |
| Kernel (dried): | a. Kernel length: 10.6 mm |
| | b. Kernel width: 7.7 mm |
| | c. Kernel thickness: 4.0 mm |
| | d. Aleurone color pattern: Solid |
| | e. Aleurone color: Yellow |
| | f. Hard endosperm color: Yellow |
| | g. Weight per 100 kernels: 20.4 g |
| Agronomic | a. Stay green (at 65 days after anthesis): 6 (Rated |
| Traits: | on a scale of 1 = worst to 9 = excellent) |
| | b. Dropped ears (at 65 days after anthesis): 0% |
| | c. Pre-anthesis root lodging: 0% |
| | d. Post-anthesis root lodging (at 65 days after |
| | anthesis): 1% |
| Disease and pest | a. Resistance to Fusarium: 7 (rated |
| resistance: | on a scale of 1 = susceptible to 9 = resistant) |
| | b. Resistance to Eyespot: 6 (rated on a scale of |
| | 1 = susceptible to 9 = resistant) |

In table 8 below, the grain percent protein (dry basis) and percent oil (dry basis) from one corn hybrid produced by using corn inbred 3B50HO of the present invention are presented. The hybrid, 3B50HO×3B603, was produced using standard hybrid breeding methods (NOT by using the TOPCROSS method). Column 1 shows the pedigree and columns 2 through 4 show the percent moisture, percent protein (dry basis) and percent oil (dry basis) respectively. HC53×LH290 and HC53×LHE323 are conventional hybrids for comparison. All the hybrids were grown in Michigan.

As shown in table 8 below, the level of protein and the level of oil in combination were unexpectedly high in the hybrids of the present invention. Normally, increased oil content in corn results in decreased protein content while increased protein content results in decreased oil content. However, the present invention has resulted in corn plants that maintain high levels of both protein and oil. Additionally, the results indicate the strong dominate effect of the high oil, high protein traits in the hybrids.

TABLE 8

| Pedigree | % Moisture | % Protein (DB) | % Oil (DB) |
|---|---|---|---|
| 3B50HO × 3B603 | 27.3 | 9.2 | 8.5 |
| 3B50HO × 3B603 | 28.9 | 9.4 | 9.0 |
| 3B50HO × 3B603 | 26.7 | 9.8 | 10.2 |
| HC53 × LH290 | 29.0 | 9.8 | 4.6 |
| HC53 × LH290 | 22.2 | 9.2 | 5.4 |
| HC53 × LH290 | 21.3 | 9.2 | 5.1 |
| HC53 × LHE323 | 23.7 | 7.9 | 4.7 |
| HC53 × LHE323 | 26.8 | 7.2 | 5.7 |
| HC53 × LHE323 | 26.9 | 7.1 | 5.8 |

### Example 4

### Inbred Corn Line 3B1174

Inbred corn line 3B1174 is a yellow field corn which provides an excellent parental line in crosses for producing high oil, high protein first generation (F₁) hybrid corn.

Some of the criteria used to select ears in various generations include: yield, percentage moisture, percentage oil, percentage protein, stalk quality, root quality, disease tolerance, late plant greenness, late season plant intactness, tip fill, barrenness, ear shape and size, seed quality, ear height, pollen shedding ability, and corn borer tolerance. During the development of the line, crosses were made to inbred testers for the purpose of estimating the line's general and specific combining ability. The inbred was then evaluated further as a line and in numerous crosses. Unexpectedly, it was found that the high oil and high protein traits appear to have a strong dominant effect in crossing and segregation. Inbred corn line 3B1174 has the following morphologic and other characteristics.

TABLE 9
VARIETY DESCRIPTION INFORMATION

| Days to 50% Flower: 97 | |
|---|---|
| GDU to 50% Flower: 1330 | |
| Days to 50% Silk: 97 | |
| GDU to 50% Silk: 1330 | |
| Kernel: | a. Kernel length: 9.9 mm |
| | b. Kernel width: 7.1 mm |
| | c. Kernel thickness: 4.7 mm |
| | d. Weight per 100 kernels: 15.08 g |

In table 10 below, the grain percent protein (dry basis) and percent oil (dry basis) from two corn hybrids produced by using corn inbred 3B1174 of the present invention are presented. These hybrids, 3B46×3B1174 and LH227RR×3B1174, were produced using standard hybrid breeding methods (NOT by using the TOPCROSS method). Column 1 shows the location, where WI is Wisconsin, MI is Michigan, and MN is Minnesota. Column 2 shows the pedigree and columns 3 through 5 show the percent moisture, percent protein (dry basis) and percent oil (dry basis) respectively. [(LH74×LH146)×LH163]-P41 is a TOPCROSS blend, while HC53×LH290 and HC53×LHE323 are conventional hybrids for comparison.

As shown in table 10 below, the level of protein and the level of oil in combination were unexpectedly high in the hybrids of the present invention. Normally, increased oil content in corn results in decreased protein content while increased protein content results in decreased oil content. However, the present invention has resulted in corn plants that maintain high levels of both protein and oil. Additionally, the results indicate the strong dominate effect of the high oil, high protein traits in the hybrids.

TABLE 10

| Loc | Pedigree | % Moisture | % Protein (DB) | % Oil (DB) |
|---|---|---|---|---|
| WI | 3B46 × 3B1174 | 8.37 | 11.84 | 6.99 |
| MI | 3B46 × 3B1174 | 27.1 | 9.0 | 8.0 |
| MI | 3B46 × 3B1174 | 27.8 | 9.0 | 8.5 |
| WI | 3B49 × 3B1174 | 7.40 | 11.87 | 8.83 |
| WI | 3B51 × 3B1174 | 9.70 | 11.35 | 7.27 |
| WI | 3B66 × 3B1174 | 6.98 | 11.63 | 7.19 |
| WI | 3B71 × 3B1174 | 6.50 | 12.36 | 6.78 |
| MI | LH227RR × 3B1174 | 26.8 | 10.6 | 7.0 |
| MI | LH227RR × 3B1174 | 28.1 | 8.1 | 6.8 |
| MI | LH227RR × 3B1174 | 30.6 | 8.2 | 7.0 |
| MI | HC53 × LHE323 | 23.7 | 7.9 | 4.7 |
| MI | HC53 × LHE323 | 26.8 | 7.2 | 5.7 |
| MI | HC53 × LHE323 | 26.9 | 7.1 | 5.8 |
| MI | HC53 × LH290 | 29.0 | 9.8 | 4.6 |
| MI | HC53 × LH290 | 22.2 | 9.2 | 5.4 |
| MI | HC53 × LH290 | 21.3 | 9.2 | 5.1 |
| WI | [(LH74 × LH146) × LH163] − P41 | 29.92 | 8.42 | 3.83 |
| WI | [(LH74 × LH1460 × LH163] − P41 | 29.42 | 8.61 | 4.23 |
| MN | [(LH74 × LH146) × LH163] − P41 | 12.37 | 9.94 | 7.61 |
| MN | [(LH74 × LH146) × LH163] − P41 | 13.72 | 10.57 | 7.22 |
| MN | [(LH74 × LH146) × LH163] − P41 | 16.04 | 10.02 | 6.91 |
| MN | [(LH74 × LH146) × LH163] − P41 | 10.89 | 11.09 | 8.34 |

### Example 5

### Experimental Inbred Corn Lines

There are a number of experimental near inbred corn lines which exhibit a high oil and high protein combination. Table 11 below shows the experimental corn lines and the percent crude protein and percent oil content for each line. As can be seen in the table, each line unexpectedly has significantly higher protein and oil content than conventional corn lines.

TABLE 11

| Corn Line | % Crude Protein | % Oil | % Dry Matter |
|---|---|---|---|
| EX184 | 13.6 | 23.8 | 89.5 |
| EX190 | 11.5 | 20.6 | 89.4 |
| EX183 | 13.5 | 22.0 | 89.5 |
| EX179 | 12.1 | 23.7 | 90.0 |
| EX229 | 14.6 | 22.5 | 89.5 |
| EX174 | 15.9 | 20.3 | 89.5 |
| EX176 | 15.0 | 20.4 | 89.3 |
| EX188 | 15.9 | 21.4 | 88.6 |
| EX231 | 16.7 | 20.4 | 89.3 |
| EX206 | 17.6 | 19.0 | 88.5 |
| EX236 | 14.2 | 21.6 | 89.3 |
| EX222 | 14.6 | 21.5 | 89.4 |

### Further Embodiments of the Invention

With the advent of molecular biological techniques that have allowed the isolation and characterization of genes that encode specific protein products, scientists in the field of plant biology developed a strong interest in engineering the genome of plants to contain and express foreign genes, or additional, or modified versions of native, or endogenous, genes (perhaps driven by different promoters) in order to alter the traits of a plant in a specific manner. Such foreign additional and/or modified genes are referred to herein collectively as “transgenes”. Over the last fifteen to twenty years several methods for producing transgenic plants have been developed, and the present invention, in particular embodiments, also relates to transformed versions of the claimed inbred line.

Plant transformation involves the construction of an expression vector which will function in plant cells. Such a vector comprises DNA comprising a gene under control of, or operatively linked to, a regulatory element (for example, a promoter). The expression vector may contain one or more such operably linked gene/regulatory element combinations. The vector(s) may be in the form of a plasmid, and can be used alone or in combination with other plasmids, to provide transformed corn plants, using transformation methods as described below to incorporate transgenes into the genetic material of the corn plant(s).

Expression Vectors for Corn Transformation—Marker Genes

Expression vectors include at least one genetic marker, operably linked to a regulatory element (a promoter, for example) that allows transformed cells containing the marker to be either recovered by negative selection, i.e., inhibiting growth of cells that do not contain the selectable marker gene, or by positive selection, i.e., screening for the product encoded by the genetic marker. Many commonly used selectable marker genes for plant transformation are well known in the transformation arts, and include, for example, genes that code for enzymes that metabolically detoxify a selective chemical agent which may be an antibiotic or a herbicide, or genes that encode an altered target which is insensitive to the inhibitor. A few positive selection methods are also known in the art.

One commonly used selectable marker gene for plant transformation is the neomycin phosphotransferase II (nptII) gene, isolated from transposon Tn5, which, when placed under the control of plant regulatory signals, confers resistance to kanamycin. Fraley et al., Proc. Natl. Acad. Sci. U.S.A., 80:4803 (1983). Another commonly used selectable marker gene is the hygromycin phosphotransferase gene which confers resistance to the antibiotic hygromycin. Vanden Elzen et al., Plant Mol. Biol., 5:299 (1985).

Additional selectable marker genes of bacterial origin that confer resistance to antibiotics include gentamycin acetyl transferase, streptomycin phosphotransferase, and aminoglycoside-3′-adenyl transferase, the bleomycin resistance determinant. Hayford et al., Plant Physiol. 86:1216 (1988); Jones et al., Mol. Gen. Genet., 210:86 (1987); Svab et al., Plant Mol. Biol. 14:197 (1990); Hille et al., Plant Mol. Biol. 7:171 (1986). Other selectable marker genes confer resistance to herbicides such as glyphosate, glufosinate or bromoxynil. Comai et al., Nature 317:741-744 (1985), Gordon-Kamm et al., Plant Cell 2:603-618 (1990) and Stalker et al., Science 242:419-423 (1988).

Other selectable marker genes for plant transformation are not of bacterial origin. These genes include, for example, mouse dihydrofolate reductase, plant 5-enolpyruvylshikimate-3-phosphate synthase and plant acetolactate synthase. Eichholtz et al., Somatic Cell Mol. Genet. 13:67 (1987), Shah et al., Science 233:478 (1986), Charest et al., Plant Cell Rep. 8:643 (1990).

Another class of marker genes for plant transformation require screening of presumptively transformed plant cells rather than direct genetic selection of transformed cells for resistance to a toxic substance such as an antibiotic. These genes are particularly useful to quantify or visualize the spatial pattern of expression of a gene in specific tissues and are frequently referred to as reporter genes because they can be fused to a gene or gene regulatory sequence for the investigation of gene expression. Commonly used genes for screening presumptively transformed cells include beta-glucuronidase (GUS), beta-galactosidase, luciferase and chloramphenicol acetyltransferase. Jefferson, R. A., Plant Mol. Biol. Rep. 5:387 (1987), Teeri et al., EMBO J. 8:343 (1989), Koncz et al., Proc. Natl. Acad. Sci U.S.A. 84:131 (1987), DeBlock et al., EMBO J. 3:1681 (1984). Another approach to the identification of relatively rare transformation events has been use of a gene that encodes a dominant constitutive regulator of the Zea mays anthocyanin pigmentation pathway. Ludwig et al., Science 247:449 (1990).

In vivo methods for visualizing GUS activity that do not require destruction of plant tissue are also available. However, these in vivo methods for visualizing GUS activity have not proven useful for recovery of transformed cells because of low sensitivity, high fluorescent backgrounds and limitations associated with the use of luciferase genes as selectable markers.

A gene encoding Green Fluorescent Protein (GFP) has been utilized as a marker for gene expression in prokaryotic and eukaryotic cells. Chalfie et al., Science 263:802 (1994). GFP and mutants of GFP may be used as screenable markers.

Expression Vectors for Corn Transformation—Promoters

Genes included in expression vectors must be driven by nucleotide sequence comprising a regulatory element, for example, a promoter. Several types of promoters are now well known in the transformation arts, as are other regulatory elements that can be used alone or in combination with promoters.

As used herein, “promoter” includes reference to a region of DNA upstream from the start of transcription and involved in recognition and binding of RNA polymerase and other proteins to initiate transcription. A “plant promoter” is a promoter capable of initiating transcription in plant cells. Examples of promoters under developmental control include promoters that preferentially initiate transcription in certain organs, such as leaves, roots, seeds and tissues such as fibers, xylem vessels, tracheids, or sclerenchyma. Such promoters are referred to as “tissue-preferred”. Promoters which initiate transcription only in certain tissue are referred to as “tissue-specific”. A “cell type” specific promoter primarily drives expression in certain cell types in one or more organs, for example, vascular cells in roots or leaves. An “inducible” promoter is a promoter which is under environmental control. Examples of environmental conditions that may effect transcription by inducible promoters include anaerobic conditions or the presence of light. Tissue-specific, tissue-preferred, cell type specific, and inducible promoters constitute the class of “non-constitutive” promoters. A “constitutive” promoter is a promoter which is active under most environmental conditions.

A. Inducible Promoters

An inducible promoter is operably linked to a gene for expression in corn. Optionally, the inducible promoter is operably linked to a nucleotide sequence encoding a signal sequence which is operably linked to a gene for expression in corn. With an inducible promoter the rate of transcription increases in response to an inducing agent.

Any inducible promoter can be used in the instant invention. See Ward et al., Plant Mol. Biol. 22:361-366 (1993). Exemplary inducible promoters include, but are not limited to, that from the ACEI system which responds to copper (Mett et al., PNAS 90:4567-4571 (1993)); In2 gene from maize which responds to benzenesulfonamide herbicide safeners Gatz et al., Mol. Gen. Genetics 243:32-38 (1994) or Tet repressor from Tn10 (Gatz et al., Mol. Gen. Genetics 227:229-237 (1991)). A particularly preferred inducible promoter is a promoter that responds to an inducing agent to which plants do not normally respond. An exemplary inducible promoter is the inducible promoter from a steroid hormone gene, the transcriptional activity of which is induced by a glucocorticosteroid hormone. Schena et al., Proc. Natl. Acad. Sci. U.S.A. 88:0421 (1991).

B. Constitutive Promoters

A constitutive promoter is operably linked to a gene for expression in corn or the constitutive promoter is operably linked to a nucleotide sequence encoding a signal sequence which is operably linked to a gene for expression in corn.

Many different constitutive promoters can be utilized in the instant invention. Exemplary constitutive promoters include, but are not limited to, the promoters from plant viruses such as the 35S promoter from CaMV (Odell et al., Nature 313:810-812 (1985)) and the promoters from such genes as rice actin (McElroy et al., Plant Cell 2:163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)) and maize H3 histone (Lepetit et al., Mol. Gen. Genetics 231:276-285 (1992) and Atanassova et al., Plant Journal 2 (3): 291-300 (1992)).

The ALS promoter, Xba1/NcoI fragment 5′ to the Brassica napus ALS3 structural gene (or a nucleotide sequence similarity to said Xba1/NcoI fragment), represents a particularly useful constitutive promoter. See PCT application WO96/30530.

C. Tissue-specific or Tissue-preferred Promoters

A tissue-specific promoter is operably linked to a gene for expression in corn. Optionally, the tissue-specific promoter is operably linked to a nucleotide sequence encoding a signal sequence which is operably linked to a gene for expression in corn. Plants transformed with a gene of interest operably linked to a tissue-specific promoter produce the protein product of the transgene exclusively, or preferentially, in a specific tissue.

Any tissue-specific or tissue-preferred promoter can be utilized in the instant invention. Exemplary tissue-specific or tissue-preferred promoters include, but are not limited to, a root-preferred promoter, such as that from the phaseolin gene (Murai et al., Science 23:476-482 (1983) and Sengupta-Gopalan et al., Proc. Natl. Acad. Sci. U.S.A. 82:3320-3324 (1985)); a leaf-specific and light-induced promoter such as that from cab or rubisco (Simpson et al., EMBO J. 4(11):2723-2729 (1985) and Timko et al., Nature 318:579-582 (1985)); an anther-specific promoter such as that from LAT52 (Twell et al., Mol. Gen. Genetics 217:240-245 (1989)); a pollen-specific promoter such as that from Zm13 or a microspore-preferred promoter such as that from apg (Twell et al., Sex. Plant Reprod. 6:217-224 (1993)).

Signal Sequences for Targeting Proteins to Subcellular Compartments

Transport of protein produced by transgenes to a subcellular compartment such as the chloroplast, vacuole, peroxisome, glyoxysome, cell wall or mitochondrion or for secretion into the apoplast is accomplished by means of operably linking the nucleotide sequence encoding a signal sequence to the 5′ and/or 3′ region of a gene encoding the protein of interest. Targeting sequences at the 5′ and/or 3′ end of the structural gene may determine, during protein synthesis and processing, where the encoded protein is ultimately compartmentalized.

The presence of a signal sequence directs a polypeptide to either an intracellular organelle or subcellular compartment or for secretion to the apoplast. Many signal sequences are known in the art. See, for example Becker et al., Plant Mol. Biol. 20:49 (1992), Knox, C., et al., Plant Mol. Biol. 9:3-17 (1987), Lerner et al., Plant Physiol. 91:124-129 (1989), Fontes et al., Plant Cell 3:483-496 (1991), Matsuoka et al., Proc. Natl. Acad. Sci. 88:834 (1991), Gould et al., J. Cell. Biol. 108:1657 (1989), Creissen et al., Plant J. 2:129 (1991), Kalderon, et al., Cell 39:499-509 (1984), Stiefel, et al., Plant Cell 2:785-793 (1990).

Foreign Protein Genes and Agronomic Genes

With transgenic plants according to the present invention, a foreign protein can be produced in commercial quantities. Thus, techniques for the selection and propagation of transformed plants, which are well understood in the art, yield a plurality of transgenic plants which are harvested in a conventional manner, and a foreign protein then can be extracted from a tissue of interest or from total biomass. Protein extraction from plant biomass can be accomplished by known methods which are discussed, for example, by Heney and Orr, Anal. Biochem. 114:92-6 (1981).

According to a preferred embodiment, the transgenic plant provided for commercial production of foreign protein is corn. In another preferred embodiment, the biomass of interest is seed. For the relatively small number of transgenic plants that show higher levels of expression, a genetic map can be generated, primarily via conventional RFLP, PCR and SSR analysis, which identifies the approximate chromosomal location of the integrated DNA molecule. For exemplary methodologies in this regard, see Glick and Thompson, Methods in Plant Molecular Biology and Biotechnology CRC Press, Boca Raton 269:284 (1993). Map information concerning chromosomal location is useful for proprietary protection of a subject transgenic plant. If unauthorized propagation is undertaken and crosses made with other germplasm, the map of the integration region can be compared to similar maps for suspect plants, to determine if the latter have a common parentage with the subject plant. Map comparisons would involve hybridizations, RFLP, PCR, SSR and sequencing, all of which are conventional techniques.

Likewise, by means of the present invention, agronomic genes can be expressed in transformed plants. More particularly, plants can be genetically engineered to express various phenotypes of agronomic interest. Exemplary genes implicated in this regard include, but are not limited to, those categorized below:

1. Genes That Confer Resistance to Pests or Disease and That Encode:

A. Plant disease resistance genes. Plant defenses are often activated by specific interaction between the product of a disease resistance gene (R) in the plant and the product of a corresponding avirulence (Avr) gene in the pathogen. A plant inbred line can be transformed with cloned resistance gene to engineer plants that are resistant to specific pathogen strains. See, for example Jones et al., Science 266:789 (1994) (cloning of the tomato Cf-9 gene for resistance to Cladosporium fulvum); Martin et al., Science 262:1432 (1993) (tomato Pto gene for resistance to Pseudomonas syringae pv. tomato encodes a protein kinase); Mindrinos et al., Cell 78:1089 (1994) (Arabidopsis RSP2 gene for resistance to Pseudomonas syringae).

B. A Bacillus thuringiensis protein, a derivative thereof or a synthetic polypeptide modeled thereon. See, for example, Geiser et al., Gene 48:109 (1986), who disclose the cloning and nucleotide sequence of a Bt alpha-endotoxin gene. Moreover, DNA molecules encoding alpha-endotoxin genes can be purchased from American Type Culture Collection, Manassas, Va., for example, under ATCC Accession Nos. 40098, 67136, 31995 and 31998.

C. A lectin. See, for example, the disclosure by Van Damme et al., Plant Molec. Biol. 24:25 (1994), who disclose the nucleotide sequences of several Clivia miniata mannose-binding lectin genes.

D. A vitamin-binding protein such as avidin. See PCT application US93/06487. The application teaches the use of avidin and avidin homologues as larvicides against insect pests.

E. An enzyme inhibitor, for example, a protease or proteinase inhibitor or an amylase inhibitor. See, for example, Abe et al., J. Biol. Chem. 262:16793 (1987) (nucleotide sequence of rice cysteine proteinase inhibitor), Huub et al., Plant Molec. Biol. 21:985 (1993) (nucleotide sequence of cDNA encoding tobacco proteinase inhibitor I), Sumitani et al., Biosci. Biotech. Biochem. 57:1243 (1993) (nucleotide sequence of Streptomyces nitrosporeus alpha-amylase inhibitor).

F. An insect-specific hormone or pheromone such as an ecdysteroid and juvenile hormone, a variant thereof, a mimetic based thereon, or an antagonist or agonist thereof. See, for example, the disclosure by Hammock et al., Nature 344:458 (1990), of baculovirus expression of cloned juvenile hormone esterase, an inactivator of juvenile hormone.

G. An insect-specific peptide or neuropeptide which, upon expression, disrupts the physiology of the affected pest. For example, see the disclosures of Pratt et al., Biochem. Biophys. Res. Comm. 163:1243 (1989) (an allostatin is identified in Diploptera puntata). See also U.S. Pat. No. 5,266,317 to Tomalski et al., who disclose genes encoding insect-specific, paralytic neurotoxins.

H. An insect-specific venom produced in nature by a snake, a wasp, etc. For example, see Pang et al., Gene 116:165 (1992), for disclosure of heterologous expression in plants of a gene coding for a scorpion insectotoxic peptide.

I. An enzyme responsible for a hyper-accumulation of a monoterpene, a sesquiterpene, a steroid, a hydroxamic acid, a phenylpropanoid derivative or another non-protein molecule with insecticidal activity.

J. An enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See PCT application WO 93/02197 in the name of Scott et al., which discloses the nucleotide sequence of a callase gene. DNA molecules which contain chitinase-encoding sequences can be obtained, for example, from the ATCC under Accession Nos. 39637 and 67152. See also Kramer et al., Insect Biochem. Molec. Biol. 23:691 (1993), who teach the nucleotide sequence of a cDNA encoding tobacco hornworm chitinase, and Kawalleck et al., Plant Molec. Biol. 21:673 (1993), who provide the nucleotide sequence of the parsley ubi4-2 polyubiquitin gene.

K. A molecule that stimulates signal transduction. For example, see the disclosure by Botella et al., Plant Molec. Biol. 24:757 (1994), of nucleotide sequences for mung bean calmodulin cDNA clones, and Griess et al., Plant Physiol. 104:1467 (1994), who provide the nucleotide sequence of a maize calmodulin cDNA clone.

L. A hydrophobic moment peptide. See PCT application WO95/16776 (disclosure of peptide derivatives of Tachyplesin which inhibit fungal plant pathogens) and PCT application WO95/18855 (teaches synthetic antimicrobial peptides that confer disease resistance).

M. A membrane permease, a channel former or a channel blocker. For example, see the disclosure of Jaynes et al., Plant Sci 89:43 (1993), of heterologous expression of a cecropin-beta, lytic peptide analog to render transgenic tobacco plants resistant to Pseudomonas solanacearum.

N. A viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See Beachy et al., Ann. Rev. Phytopathol. 28:451 (1990). Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. Id.

O. An insect-specific antibody or an immunotoxin derived therefrom. Thus, an antibody targeted to a critical metabolic function in the insect gut would inactivate an affected enzyme, killing the insect.

P. A virus-specific antibody. See, for example, Tavladoraki et al., Nature 366:469 (1993), who show that transgenic plants expressing recombinant antibody genes are protected from virus attack.

Q. A developmental-arrestive protein produced in nature by a pathogen or a parasite. Thus, fungal endo-alpha-1,4-D-polygalacturonases facilitate fungal colonization and plant nutrient release by solubilizing plant cell wall homo-alpha-1,4-D-galacturonase. See Lamb et al., BioTechnology 10:1436 (1992). The cloning and characterization of a gene which encodes a bean endopolygalacturonase-inhibiting protein is described by Toubart et al., Plant J. 2:367 (1992).

R. A developmental-arrestive protein produced in nature by a plant. For example, Logemann et al., BioTechnology 10:305 (1992), have shown that transgenic plants expressing the barley ribosome-inactivating gene have an increased resistance to fungal disease.

2. Genes That Confer Resistance to an Herbicide, For Example:

A. An herbicide that inhibits the growing point or meristem, such as an imidazolinone or a sulfonylurea. Exemplary genes in this category code for mutant ALS and AHAS enzyme as described, for example, by Lee et al., EMBO J. 7:1241 (1988), and Miki et al., Theor. Appl. Genet. 80:449 (1990), respectively.

B. Glyphosate (resistance conferred by mutant 5-enolpyruvylshikimate-3-phosphate synthase (EPSP) and aroA genes, respectively) and other phosphono compounds such as glufosinate (phosphinothricin acetyl transferase (PAT) and Streptomyces hygroscopicus PAT, bar, genes), and pyridinoxy or phenoxy propionic acids and cyclohexones (ACCase inhibitor-encoding genes). See, for example, U.S. Pat. No. 4,940,835 to Shah, et al., which discloses the nucleotide sequence of a form of EPSP which can confer glyphosate resistance. A DNA molecule encoding a mutant aroA gene can be obtained under ATCC accession number 39256, and the nucleotide sequence of the mutant gene is disclosed in U.S. Pat. No. 4,769,061 to Comai. European patent application No. 0 333 033 to Kumada et al., and U.S. Pat. No. 4,975,374 to Goodman et al., disclose nucleotide sequences of glutamine synthetase genes which confer resistance to herbicides such as L-phosphinothricin. The nucleotide sequence of a PAT gene is provided in European application No. 0 242 246 to Leemans et al. DeGreef et al., BioTechnology 7:61 (1989), describe the production of transgenic plants that express chimeric bar genes coding for PAT activity. Exemplary of genes conferring resistance to phenoxy propionic acids and cyclohexones, such as sethoxydim and haloxyfop are the Acc1-S1, Acc1-S2 and Acc1-S3 genes described by Marshall et al., Theor. Appl. Genet. 83:435 (1992).

C. An herbicide that inhibits photosynthesis, such as a triazine (psba and gs+ genes) or a benzonitrile (nitrilase gene). Przibilla et al., Plant Cell 3:169 (1991), describe the transformation of Chlamydomonas with plasmids encoding mutant psbA genes. Nucleotide sequences for nitrilase genes are disclosed in U.S. Pat. No. 4,810,648 to Stalker, and DNA molecules containing these genes are available under ATCC Accession Nos. 53435, 67441, and 67442. Cloning and expression of DNA coding for a glutathione S-transferase is described by Hayes et al., Biochem. J. 285:173 (1992).

3. Genes that Confer or Contribute to a Value-Added Trait, Such as:

A. Modified fatty acid metabolism, for example, by transforming a plant with an antisense gene of stearyl-ACP desaturase to increase stearic acid content of the plant. See Knutzon et al., Proc. Natl. Acad. Sci. U.S.A. 89:2624 (1992)

B. Increased resistance to high light stress such as photo-oxidative damages, for example by transforming a plant with a gene coding for a protein of the Early Light Induced Protein family (ELIP) as described in WO 03074713 in the name of Biogemma.

C. Modified carbohydrate composition effected, for example, by transforming plants with a gene coding for an enzyme that alters the branching pattern of starch. See Shiroza et al., J. Bact. 170:810 (1988) (nucleotide sequence of Streptococcus mutants fructosyltransferase gene), Steinmetz et al., Mol. Gen. Genet. 20:220 (1985) (nucleotide sequence of Bacillus subtilis levansucrase gene), Pen et al., BioTechnology 10:292 (1992) (production of transgenic plants that express Bacillus lichenifonnis alpha-amylase), Elliot et al., Plant Molec. Biol. 21:515 (1993) (nucleotide sequences of tomato invertase genes), Søgaard et al., J. Biol. Chem. 268:22480 (1993) (site-directed mutagenesis of barley alpha-amylase gene), and Fisher et al., Plant Physiol. 102:1045 (1993) (maize endosperm starch branching enzyme II).

D. Increased resistance/tolerance to water stress or drought, for example, by transforming a plant to create a plant having a modified content in ABA-Water-Stress-Ripening-Induced proteins (ARS proteins) as described in WO 0183753 in the name of Biogemma, or by transforming a plant with a nucleotide sequence coding for a phosphoenolpyruvate carboxylase as shown in WO02081714. The tolerance of corn to drought can also be increased by an overexpression of phosphoenolpyruvate carboxylase (PEPC-C4), obtained, for example from sorghum.

E. Increased content of cysteine and glutathione, useful in the regulation of sulfur compounds and plant resistance against various stresses such as drought, heat or cold, by transforming a plant with a gene coding for an Adenosine 5′ Phosphosulfate as shown in WO 0149855.

F. Increased nutritional quality, for example, by introducing a zein gene which genetic sequence has been modified so that its protein sequence has an increase in lysine and proline. The increased nutritional quality can also be attained by introducing into the maize plant an albumin 2S gene from sunflower that has been modified by the addition of the KDEL peptide sequence to keep and accumulate the albumin protein in the endoplasmic reticulum.

G. Decreased phytate content. 1) Introduction of a phytase-encoding gene would enhance breakdown of phytate, adding more free phosphate to the transformed plant. For example, see Van Hartingsveldt et al., Gene 127:87 (1993), for a disclosure of the nucleotide sequence of an Aspergillus niger phytase gene. 2) A gene could be introduced that reduced phytate content. In maize, this, for example, could be accomplished, by cloning and then reintroducing DNA associated with the single allele which is responsible for maize mutants characterized by low levels of phytic acid. See Raboy et al., Maydica 35:383 (1990).

Methods for Corn Transformation

Numerous methods for plant transformation have been developed, including biological and physical plant transformation protocols. See, for example, Miki et al., “Procedures for Introducing Foreign DNA into Plants” in Methods in Plant Molecular Biology and Biotechnology, Glick B. R. and Thompson, J. E. Eds. (CRC Press, Inc., Boca Raton, 1993) pages 67-88. In addition, expression vectors and in vitro culture methods for plant cell or tissue transformation and regeneration of plants are available. See, for example, Gruber et al., “Vectors for Plant Transformation” in Methods in Plant Molecular Biology and Biotechnology, Glick B. R. and Thompson, J. E. Eds. (CRC Press, Inc., Boca Raton, 1993) pages 89-119.

A. Agrobacterium-mediated Transformation

One method for introducing an expression vector into plants is based on the natural transformation system of Agrobacterium. See, for example, Horsch et al., Science 227:1229 (1985). A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. See, for example, Kado, C. I., Crit. Rev. Plant Sci. 10:1 (1991). Descriptions of Agrobacterium vector systems and methods for Agrobacterium-mediated gene transfer are provided by Gruber et al., supra, Miki et al., supra, and Moloney et al., Plant Cell Reports 8:238 (1989). See also, U.S. Pat. No. 5,591,616 issued Jan. 7, 1997.

B. Direct Gene Transfer

Despite the fact the host range for Agrobacterium-mediated transformation is broad, some major cereal crop species and gymnosperms have generally been recalcitrant to this mode of gene transfer, even though some success has recently been achieved in rice and corn. Hiei et al., The Plant Journal 6:271-282 (1994) and U.S. Pat. No. 5,591,616 issued Jan. 7, 1997. Several methods of plant transformation collectively referred to as direct gene transfer have been developed as an alternative to Agrobacterium-mediated transformation.

A generally applicable method of plant transformation is microprojectile-mediated transformation wherein DNA is carried on the surface of microprojectiles measuring 1 to 4 micron. The expression vector is introduced into plant tissues with a biolistic device that accelerates the microprojectiles to speeds of 300 to 600 m/s which is sufficient to penetrate plant cell walls and membranes. Sanford et al., Part. Sci. Technol. 5:27 (1987), Sanford, J. C., Trends Biotech. 6:299 (1988), Klein et al., Biotechnology 6:559-563 (1988), Sanford, J. C., Physiol Plant 7:206 (1990), Klein et al., Biotechnology 10:268 (1992). In corn, several target tissues can be bombarded with DNA-coated microprojectiles in order to produce transgenic plants, including, for example, callus (Type I or Type II), immature embryos, and meristematic tissue.

Another method for physical delivery of DNA to plants is sonication of target cells. Zhang et al., BioTechnology 9:996 (1991). Alternatively, liposome and spheroplast fusion have been used to introduce expression vectors into plants. Deshayes et al., EMBO J., 4:2731 (1985), Christou et al., Proc Natl. Acad. Sci. U.S.A. 84:3962 (1987). Direct uptake of DNA into protoplasts using CaCl₂ precipitation, polyvinyl alcohol or poly-L-ornithine has also been reported. Hain et al., Mol. Gen. Genet. 199:161 (1985) and Draper et al., Plant Cell Physiol. 23:451 (1982). Electroporation of protoplasts and whole cells and tissues have also been described. D'Halluin et al., Plant Cell 4:1495-1505 (1992) and Spencer et al., Plant Mol. Biol. 24:51-61 (1994).

Following transformation of corn target tissues, expression of the above-described selectable marker genes allows for preferential selection of transformed cells, tissues and/or plants, using regeneration and selection methods now well known in the art.

The foregoing methods for transformation would typically be used for producing a transgenic inbred line. The transgenic inbred line could then be crossed, with another (non-transformed or transformed) inbred line, in order to produce a new transgenic inbred line. Alternatively, a genetic trait which has been engineered into a particular corn line using the foregoing transformation techniques could be moved into another line using traditional backcrossing techniques that are well known in the plant breeding arts. For example, a backcrossing approach could be used to move an engineered trait from a public, non-elite inbred line into an elite inbred line, or from an inbred line containing a foreign gene in its genome into an inbred line or lines which do not contain that gene. As used herein, “crossing” can refer to a simple X by Y cross, or the process of backcrossing, depending on the context.

When the term inbred corn plant is used in the context of the present invention, this also includes any inbred corn plant where one or more desired trait has been introduced through backcrossing methods, whether such trait is a naturally occurring one or a transgenic one. Backcrossing methods can be used with the present invention to improve or introduce one or more characteristic into the inbred. The term backcrossing as used herein refers to the repeated crossing of a hybrid progeny back to one of the parental corn plants for that inbred. The parental corn plant which contributes the gene or the genes for the desired characteristic is termed the nonrecurrent or donor parent. This terminology refers to the fact that the nonrecurrent parent is used one time in the backcross protocol and therefore does not recur. The parental corn plant to which the gene or genes from the nonrecurrent parent are transferred is known as the recurrent parent as it is used for several rounds in the backcrossing protocol (Fehr, 1987).

In a typical backcross protocol, the original inbred of interest (recurrent parent) is crossed to a second inbred (nonrecurrent parent) that carries the gene or genes of interest to be transferred. The resulting progeny from this cross are then crossed again to the recurrent parent and the process is repeated until a corn plant is obtained wherein all the desired morphological and physiological characteristics of the recurrent parent are recovered in the converted plant, generally determined at a 5% significance level when grown in the same environmental conditions, in addition to the gene or genes transferred from the nonrecurrent parent. It should be noted that some, one, two, three or more, self-pollination and growing of a population might be included between two successive backcrosses. Indeed, an appropriate selection in the population produced by the self-pollination, i.e. selection for the desired trait and physiological and morphological characteristics of the recurrent parent might be equivalent to one, two or even three additional backcrosses in a continuous series without rigorous selection, saving time, money and effort for the breeder.

A non limiting example of such a protocol would be the following: a) the first generation F₁. produced by the cross of the recurrent parent A by the donor parent B is backcrossed to parent A, b) selection is practiced for the plants having the desired trait of parent B, c) selected plant are self-pollinated to produce a population of plants where selection is practiced for the plants having the desired trait of parent B and physiological and morphological characteristics of parent A, d) the selected plants are backcrossed one, two, three, four, fifth or more times to parent A to produce selected backcross progeny plants comprising the desired trait of parent B and physiological and morphological characteristics of parent A. Step c) may or may not be repeated and included between the backcrosses of step d.

The selection of a suitable recurrent parent is an important step for a successful backcrossing procedure. The goal of a backcross protocol is to alter or substitute one or more trait(s) or characteristic(s) in the original inbred. To accomplish this, a gene or genes of the recurrent inbred is modified or substituted with the desired gene or genes from the nonrecurrent parent, while retaining essentially all of the rest of the desired genetic, and therefore the desired physiological and morphological, constitution of the original inbred. The choice of the particular nonrecurrent parent will depend on the purpose of the backcross; one of the major purposes is to add some commercially desirable, agronomically important trait(s) to the plant. The exact backcrossing protocol will depend on the characteristic(s) or trait(s) being altered to determine an appropriate testing protocol. Although backcrossing methods are simplified when the characteristic being transferred is a single gene and dominant allele, multiple genes and recessive allele(s) may also be transferred and therefore, backcross breeding is by no means restricted to character(s) governed by one or a few genes. In fact the number of genes might be less important that the identification of the character(s) in the segregating population. In this instance it may then be necessary to introduce a test of the progeny to determine if the desired characteristic(s) has been successfully transferred. Such tests encompass not only visual inspection and simple crossing, but also follow up of the characteristic(s) through genetically associated markers and molecular assisted breeding tools. For example, selection of progeny containing the transferred trait is done by direct selection, visual inspection for a trait associated with a dominant allele, while the selection of progeny for a trait that is transferred via a recessive allele, such as the waxy starch characteristic, require selfing the progeny to determine which plant carry the recessive allele(s).

Many single gene traits have been identified that are not regularly selected for in the development of a new inbred but that can be improved by backcrossing techniques. Single gene traits may or may not be transgenic, i.e. they may be naturally present in the non recurrent parent, examples of these traits include but are not limited to, male sterility, waxy starch, amylose starch, herbicide resistance, resistance for bacterial, fungal, or viral disease, insect resistance, male fertility, water stress tolerance, enhanced nutritional quality, industrial usage, increased digestibility yield stability and yield enhancement. An example of gene controlling resistance to rust fungus would be the Rp1D gene, which as other Rp resistance prevents P. sorghi from producing spores. This Rp1D gene was usually preferred over the other Rp genes because it was widely effective against all races of rust, but the emergence of new races lead to use other Rp genes comprising for example the Rp1E, Rp1G, Rp1I, Rp1K or “compound” genes which combine two or more Rp genes including Rp1GI, Rp1GDJ, etc. These genes are generally inherited through the nucleus. Some known exceptions to this are the genes for male sterility, some of which are inherited cytoplasmically, but still act as single gene traits. Several of these single gene traits are described in U.S. Pat. Nos. 5,777,196; 5,948,957 and 5,969,212, the disclosures of which are specifically hereby incorporated by reference.

In 1981, the backcross method of breeding accounted for 17% of the total breeding effort for inbred line development in the United States, according to, Hallauer, A. R. et al. (1988) “Corn Breeding” Corn and Corn Improvement, No. 18, pp. 463-481.

The backcross breeding method provides a precise way of improving varieties that excel in a large number of attributes but are deficient in a few characteristics. (Page 150 of Allard (1960), published by John Wiley & Sons, Inc, “Principles of Plant Breeding”). The method makes use of a series of backcrosses to the variety to be improved during which the character or the characters in which improvement is sought is maintained by selection. At the end of the backcrossing, the gene or genes being transferred, unlike all other genes, will be heterozygous. Selfing after the last backcross produces homozygosity for this gene pair(s) and, coupled with selection, will result in a variety with exactly the adaptation, yielding ability and quality characteristics of the recurrent parent but superior to that parent in the particular characteristic(s) for which the improvement program was undertaken. Therefore, this method provides the plant breeder with a high degree of genetic control of his work.

The method is scientifically exact because the morphological and agricultural features of the improved variety could be described in advance and because the same variety could, if it were desired, be bred a second time by retracing the same steps (Briggs, “Breeding wheats resistant to bunt by the backcross method”, 1930 Jour. Amer. Soc. Agron., 22: 289-244).

Backcrossing is a powerful mechanism for achieving homozygosity and any population obtained by backcrossing must rapidly converge on the genotype of the recurrent parent. When backcrossing is made the basis of a plant breeding program, the genotype of the recurrent parent will be modified only with regards to genes being transferred, which are maintained in the population by selection.

Successful backcrosses are, for example, the transfer of stem rust resistance from “Hope” wheat to “Bart” wheat and even pursuing the backcrosses with the transfer of bunt resistance to create “Bart 38”, having both resistances. Also highlighted by Allard is the successful transfer of mildew, leaf spot and wilt resistances in “California Common” alfalfa to create “Caliverde”. This new “Caliverde” variety produced through the backcross process is indistinguishable from “California Common” except for its resistance to the three named diseases.

One of the advantages of the backcross method is that the breeding program can be carried out in almost every environment that will allow the development of the character being transferred.

The backcross technique is not only desirable when breeding for disease resistance but also for the adjustment of morphological characters, colour characteristics and simply inherited quantitative characters such as earliness, plant height and seed size and shape. In this regard, a medium grain type variety, “Calady”, has been produced by Jones and Davis. In dealing with quantitative characteristics, they selected the donor parent with the view of sacrificing some of the intensity of the character for which it was chosen, i.e. grain size. “Lady Wright”, a long grain variety was used as the donor parent and “Coloro”, a short grain one as the recurrent parent. After four backcrosses, the medium grain type variety “Calady” was produced.

The corn inbreds and hybrids of the present invention can be used by a skilled plant breeder to produce additional high oil and high protein corn lines using any of the various standard breeding methods.

### Deposit Information

A deposit of a mixture of corn inbreds 3B703 (703), 3B603 (603), 3B50HO (3B50) and 3B1174 (1174) has been made with the American Type Culture Collection (ATCC), 10801 University Boulevard, Manassas, Va. 20110. The date of deposit was Jul. 15, 2005. The deposit of 2,500 seeds was taken from the same deposit maintained by Brownseed Genetics, Inc. since prior to the filing date of this application. Access to this deposit will be available during the pendency of this application to persons determined by the Commissioner of Patent and Trademarks to be entitled thereto under 37 CRF 1.14 and 35 USC 122. Upon allowance of any claims in this application, all restrictions on the availability to the public of the variety will be irrevocably removed by affording access to a deposit of at least 2,500 seeds of the same variety with the American Type Culture Collection (ATCC), 10801 University Boulevard, Manassas, Va. 20110. The ATCC accession number is PTA-6869. The deposit will be maintained in the depository for a period of 30 years, or 5 years after the last request, or for the effective life of the patent, whichever is longer, and will be replaced as necessary during that period.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. A corn seed having a protein content between 7.5% and 17.9% and having an oil content between 9.0% and 23.9%.

2. A corn seed having a protein content between 9.0% and 17.9% and having an oil content between 6.5% and 23.9%.

3. The seed of claim 1, wherein the protein content is between 9.0% and 17.9%.

4. The seed of claim 2, wherein the oil content is between 9.0% and 23.9%.

5. A corn plant, or a part thereof, produced by growing the seed of claim 1.

6. A tissue culture of regenerable cells produced from the plant of claim 5, wherein cells of the tissue culture are produced from a plant part selected from the group consisting of leaves, pollen, embryos, roots, root tips, anthers, silks, flowers, kernels, ears, cobs, husks, seeds and stalks.

7. A corn plant regenerated from the tissue culture of claim 6.

8. A method for producing a hybrid corn seed wherein the method comprises crossing a first parent corn plant with a second parent corn plant and harvesting the resultant hybrid corn seed wherein said first parent corn plant or said second parent corn plant is the plant of claim 5.

9. The seed of claim 1, wherein use of the seed is selected from the group consisting of production of ethanol, a fuel in combustion, animal feed and as an ingredient in food intended for human consumption.

10. A corn plant, or a part thereof, produced by growing the seed of claim 2.

11. A tissue culture of regenerable cells produced from the plant of claim 10, wherein cells of the tissue culture are produced from a plant part selected from the group consisting of leaves, pollen, embryos, roots, root tips, anthers, silks, flowers, kernels, ears, cobs, husks, seeds and stalks.

12. A corn plant regenerated from the tissue culture of claim 11.

13. A method for producing a hybrid corn seed wherein the method comprises crossing a first parent corn plant with a second parent corn plant and harvesting the resultant hybrid corn seed wherein said first parent corn plant or said second parent corn plant is the plant of claim 10.

14. The seed of claim 2, wherein the use of the seed is selected from the group consisting of the production of ethanol, a fuel in combustion, for animal feed and as an ingredient in food intended for human consumption.

15. A corn seed having a protein content of about 11.5% to about 17.6%.

16. The seed of claim 15, wherein the use of the seed is selected from the group consisting of production of ethanol, a fuel in combustion, animal feed and as an ingredient in food intended for human consumption.

17. A method of introducing a desired trait into the plant of claim 5 wherein the method comprises:
(a) crossing the plant of claim 5, with a plant of a corn inbred that comprises a desired trait to produce progeny plants wherein the desired trait is selected from the group consisting of male sterility, herbicide resistance, insect resistance, modified fatty acid metabolism, modified carbohydrate metabolism, modified starch metabolism and resistance to bacterial disease, fungal disease or viral disease;
(b) selecting one or more progeny plants that have the desired trait to produce selected progeny plants;
(c) crossing the selected progeny plants with the plant of claim 5 to produce backcross progeny plants;
(d) selecting for backcross progeny plants that have the desired trait and the physiological and morphological characteristics of plant of claim 5 to produce selected backcross progeny plants; and
(e) repeating steps (c) and (d) to produce selected third or higher backcross progeny plants that comprise the desired trait and physiological and morphological characteristics of plant of claim 5 when grown in the same environmental conditions.

18. A method of introducing a desired trait into the plant of claim 10 wherein the method comprises:
(a) crossing the plant of claim 10, with a plant of a corn inbred that comprises a desired trait to produce progeny plants wherein the desired trait is selected from the group consisting of male sterility, herbicide resistance, insect resistance, modified fatty acid metabolism, modified carbohydrate metabolism, modified starch metabolism and resistance to bacterial disease, fungal disease or viral disease;
(b) selecting one or more progeny plants that have the desired trait to produce selected progeny plants;
(c) crossing the selected progeny plants with the plant of claim 10 to produce backcross progeny plants;
(d) selecting for backcross progeny plants that have the desired trait and the physiological and morphological characteristics of plant of claim 10 to produce selected backcross progeny plants; and
(e) repeating steps (c) and (d) to produce selected third or higher backcross progeny plants that comprise the desired trait and physiological and morphological characteristics of plant of claim 10 when grown in the same environmental conditions.

19. The seed of claim 1, wherein said seed when used as a fuel in combustion produces between 8450 Btu/lb to 9100 Btu/lb.

20. The seed of claim 2, wherein said seed when used as a fuel in combustion produces between 8450 Btu/lb to 9100 Btu/lb.
